(19) United States
(12) Patent Application Publication
(10) Pub. No.: US 2006/0235487 A1
(43) Pub. Date: Oct. 19, 2006

(54) Method and system for treatment of eating disorders by means of neuro-electrical coded signals

(76) Inventors: Dennis Meyer, Albuquerque, NM (US); Dennis P. Vik, Albuquerque, NM (US)

Correspondence Address: Ralph C. Francis;Francis Law Group, 1942 Embarcadero, Oakland, CA, 94606, US

(21) Appl. No.: 11/431,869

(22) Filed: May 9, 2006

Related U.S. Application Data
(63) Continuation-in-part of application No. 11/134,767, filed on May 20, 2005.
Application No. 11/134,767 is a continuation-in-part of application No. 11/125,480, filed on May 9, 2005.
Application No. 11/125,480 is a continuation-in-part of application No. 10/847,738, filed on May 17, 2004, now Pat. No. 6,937,903.
(60) Provisional application No. 60/471,104, filed on May 16, 2003.

(51) Int. Cl. A61N 1/18

(52) U.S. Cl. 607/58

(57) ABSTRACT

A method for treating eating disorders comprising the steps of generating at least one confounding gastrointestinal signal and at least one satiety or hunger signal that substantially corresponds to a neuro-electrical signal that is generated in a body and produces a gastrointestinal effect in the body, sensing food intake in a subject over at least a first period of time, and transmitting the confounding gastrointestinal signal and a satiety or hunger signal to the subject if the food intake of the subject exceeds or falls below a predetermined threshold level during the first period of time.

### CROSS-REFERENCE TO RELATED APPLICATIONS

This application is a continuation-in-part of U.S. application Ser. No. 11/134,767, filed May 20, 2005, which in turn is a continuation-in-part of U.S. application Ser. No. 11/125,480, filed May 9, 2005, which in turn is a continuation-in-part of U.S. application Ser. No. 10/847,738, filed May 17, 2004, which claims the benefit of U.S. Provisional Application No. 60/471,104, filed May 16, 2003.

### FIELD OF THE PRESENT INVENTION

The present invention relates generally to medical methods and systems for treating eating disorders. More particularly, the invention relates to a method and system for treatment of eating disorders by means of neuro-electrical coded signals.

### BACKGROUND OF THE INVENTION

As is well known in the art, the brain regulates (or controls) feeding behavior and gastrointestinal function via electrical signals (i.e., action potentials), which are transmitted through the nervous system. The term gastrointestinal function, as used herein, means the operation of all organs and systems involved in the process of digestion, including the alimentary canal, esophagus, stomach, small and large intestines, colon, rectum, anus, muscles affecting these organs, and the nervous system associated therewith.

It is also well known in the art that an organism employs two main cues to regulate food intake; short term cues that regulate the size of individual meals and long-term cues that regulate overall body weight. Short-term cues consist primarily of chemical properties of the food that act in the mouth to stimulate feeding behavior and in the gastrointestinal system and liver to inhibit food intake. Short-term action potentials or “gastrointestinal signals”, which are associated with (or provided by) the short-term cues, are transmitted through the nervous system and impinge on the hypothalamus through visceral afferent pathways, communicating primarily with the lateral hypothalamic regions (or satiety centers) of the brain.

The effectiveness of short-term cues is modulated by long-term signals that reflect body weight. These long-term signals are similarly transmitted through the nervous system.

One important long-term signal is the peptide leptin, which is secreted from fat storage cells (i.e. adipocytes). By means of this signal, body weight is kept reasonably constant over a broad range of activity and diet.

As indicated, the short and long-term signals are transmitted through the nervous system. Indeed, as discussed in detail herein, the vagus nerve plays a significant role in mediating afferent information from the stomach to the satiety centers of the brain.

As is known in the art, the nervous system includes two components: the central nervous system, which comprises the brain and the spinal cord, and the peripheral nervous system, which generally comprises groups of nerve cells (i.e., neurons) and peripheral nerves that lie outside the brain and spinal cord. The two systems are anatomically separate, but functionally interconnected.

As indicated, the peripheral nervous system is constructed of nerve cells (or neurons) and glial cells (or glia), which support the neurons. Operative neuron units that carry signals from the brain are referred to as “efferent” nerves. “Afferent” nerves are those that carry sensor or status information to the brain.

A typical neuron includes four morphologically defined regions: (i) cell body, (ii) dendrites, (iii) axon and (iv) presynaptic terminals. The cell body (soma) is the metabolic center of the cell. The cell body contains the nucleus, which stores the genes of the cell, and the rough and smooth endoplasmic reticulum, which synthesizes the proteins of the cell.

The cell body typically includes two types of outgrowths (or processes); the dendrites and the axon. Most neurons have multiple dendrites; these branch out in tree-like fashion and serve as the main apparatus for receiving signals from other nerve cells.

The axon is the main conducting unit of the neuron. The axon is capable of conveying electrical signals along distances that range from as short as 0.1 mm to as long as 2 m. Many axons split into several branches, thereby conveying information to different targets.

Near the end of the axon, the axon is divided into fine branches that make contact with other neurons. The point of contact is referred to as a synapse. The cell transmitting a signal is called the presynaptic cell. The cell receiving the signal is referred to as the postsynaptic cell. Specialized swellings on the axon's branches (i.e., presynaptic terminals) serve as the transmitting site in the presynaptic cell.

Most axons terminate near a postsynaptic neuron's dendrites. However, communication can also occur at the cell body or, less often, at the initial segment or terminal portion of the axon of the postsynaptic cell.

As with other physiologic systems, the gastrointestinal (“GI”) tract is subject to regulation by electrical signals that are transmitted through the nervous system. As discussed above, feeding behavior or food intake is also subject to regulation by electrical short-term and long-term signals that are transmitted through the nervous system.

The electrical signals transmitted along an axon to regulate food intake and gastrointestinal function, referred to as action potentials, are rapid and transient “all-or-none” nerve impulses. Action potentials typically have an amplitude of approximately 100 millivolts (mV) and a duration of approximately 1 msec. Action potentials are conducted along the axon, without failure or distortion, at rates in the range of approximately 1-100 meters/sec. The amplitude of the action potential remains constant throughout the axon, since the impulse is continually regenerated as it traverses the axon.

A “neurosignal” is a composite signal that includes many action potentials. The neurosignal also includes an instruction set for proper organ and/or system function. A neurosignal that controls gastrointestinal function would thus include an instruction set for the muscles of the colon and anus to perform an efficient elimination or retention of a stool bolus, including information regarding initial muscle tension, degree (or depth) of muscle movement, etc.

Neurosignals or “neuro-electrical coded signals” are thus codes that contain complete sets of information for control of organ function. As set forth in Co-Pending application Ser. No. 11/125,480, filed May 9, 2005, once these neuro-electrical signals have been isolated, recorded and standardized, a nerve-specific neuro-electrical signal or instruction can be generated and transmitted to a subject to control gastrointestinal function and, hence, treat a multitude of digestive system diseases and disorders, including, but not limited to, bowel (or fecal) incontinence, constipation and diarrhea. In accordance with the present invention, discussed in detail herein, a neuro-electrical signal can also be generated and transmitted to a subject to regulate food intake and, hence, treat various eating disorders, including, but not limited to, compulsive overeating and obesity, bulimia and anorexia nervosa.

The increasing prevalence of eating disorders, particularly obesity, in adults (and children) is one of the most serious and widespread health problems facing the world community. It is estimated that currently in America 55% of adults are obese and 20% of teenagers are either obese or significantly overweight. Additionally, 6% of the total population of the United States is morbidly obese.

This data is alarming for numerous reasons, not the least of which is it indicates an obesity epidemic. Many health experts believe that obesity is the first or second leading cause of preventable deaths in the United States, with cigarette smoking either just lagging or leading.

It is the consequences of being overweight that are most alarming. Obesity is asserted to be the cause of approximately eighty percent of adult onset diabetes in the United States, and of ninety percent of sleep apnea cases. Obesity is also a substantial risk factor for coronary artery disease, stroke, chronic venous abnormalities, numerous orthopedic problems and esophageal reflux disease. More recently, researchers have documented a link between obesity, infertility and miscarriages, as well as post menopausal breast cancer.

Despite these statistics, treatment options for obese people are limited. Classical models combining nutritional counseling with exercise and education have not led to long term success for very many patients. Use of liquid diets and pharmaceutical agents may result in weight loss which, however, is only rarely sustained. Surgical procedures that cause either gastric restriction or malabsorption have been, collectively, the most successful long-term remedy for severe obesity. However, this type of surgery involves a major operation, can lead to emotional problems, and cannot be modified readily as patient needs demand or change.

Various “electrical stimulation” apparatus, systems and methods have also been employed to treat compulsive overeating and obesity. The noted systems and methods typically include the transmission of a pre-programmed electrical pulse or signal to a subject to induce a satiety effect, e.g., feeling of fullness. Illustrative are the systems and methods disclosed in U.S. Pat. Nos. 5,263,480 and 6,587,719, and U.S. Pat. Application Publications 2005/0033376 A1 and 2004/0024428 A1.

A major drawback associated with the “electrical stimulation” systems and methods disclosed in the noted patents and publications, as well as most known systems, is that the stimulus signals that are generated and transmitted to a subject are “user determined” and, in many instances “device determinative” (e.g., neurostimulator). Since the “stimulus signals” are not related to or representative of the signals that are generated in the body, the stimulus levels required to achieve the desired satiety effect are often excessive and can elicit deleterious side effects.

It would thus be desirable to provide a method and system for treating eating disorders that includes means for generating and transmitting neuro-electrical (or “gastrointestinal”) signals to a subject that substantially correspond to neuro-electrical coded signals that are generated in the body and produce or induce a gastrointestinal effect in the body, i.e. a feeling of fullness or sensation of hunger.

It is therefore an object of the present invention to provide a method and system for treating eating disorders that overcomes the drawbacks associated with prior art methods and systems for treating eating disorders.

It is another object of the invention to provide a method and system for treating eating disorders that includes means for recording neuro-electrical signals that are generated in the body and produce a gastrointestinal effect in the body.

It is another object of the invention to provide a method and system for treating eating disorders that includes means for generating gastrointestinal signals that substantially correspond to neuro-electrical signals that are generated in the body and produce a gastrointestinal effect in the body.

It is another object of the invention to provide a method and system for treating eating disorders that includes means for monitoring food intake or consumption of a subject.

It is another object of the invention to provide a method and system for treating eating disorders that includes means for transmitting gastrointestinal signals to a subject's body that substantially correspond to neuro-electrical signals that are generated in the body and produce a gastrointestinal effect in the body in response to the subject's food intake exceeding or falling below a predetermined threshold level.

It is another object of the invention to provide a method and system for treating eating disorders that includes means for timed transmission of gastrointestinal signals to a subject that substantially correspond to neuro-electrical signals that are generated in the body and produce a gastrointestinal effect in the body.

It is another object of the invention to provide a method and system for treating eating disorders that includes means for manual transmission of gastrointestinal signals to a subject that substantially correspond to neuro-electrical signals that are generated in the body and produce a gastrointestinal effect in the body.

It is another object of the invention to provide a method and system for treating eating disorders that includes means for generating and transmitting inverted satiety signals to a subject's body to restrict the transfer of afferent information associated with a feeling of fullness to the subject's brain.

It is another object of the invention to provide a method and system for treating eating disorders that includes means for generating and transmitting inverted hunger signals to a subject's body to restrict the transfer of afferent information associated with a sensation of hunger to the subject's brain.

It is yet another object of the invention to provide a method and system for treating eating disorders that includes means for generating and transmitting confounding gastrointestinal signals to a subject's body to restrict or control the transfer of afferent information associated with a sensation of fullness and/or hunger to the satiety centers of the subject's brain.

### SUMMARY OF THE INVENTION

In accordance with the above objects and those that will be mentioned and will become apparent below, in one embodiment of the invention, the method for treating eating disorders includes the steps of (i) generating at least one inverted hunger signal, the inverted hunger signal having at least a portion thereof that is substantially similar to a negative mirror image of a portion of at least one neuro-electrical signal that is generated in a body and induces a sensation of hunger in the body and (ii) transmitting the inverted hunger signal to the subject.

In another embodiment of the invention, the method for treating eating disorders includes the steps of (i) generating at least one inverted hunger signal, the inverted hunger signal having at least a portion thereof that is substantially similar to a negative mirror image of a portion of at least one neuro-electrical signal that is generated in a body and induces a sensation of hunger in the body, (ii) generating at least one satiety signal that substantially corresponds to at least one neuro-electrical signal that is generated in a body and induces a feeling of fullness in the body, and (iii) transmitting the inverted hunger signal and satiety signal to the subject.

In another embodiment of the invention, the method for treating eating disorders includes the steps of (i) generating at least one inverted hunger signal, the inverted hunger signal having at least a portion thereof that is substantially similar to a negative mirror image of a portion of at least one neuro-electrical signal that is generated in a body and induces a sensation of hunger in the body, (ii) generating at least one confounding gastrointestinal signal that is adapted to confound neuro-electrical signals that are generated in the body and induce a sensation of hunger, (iii) generating at least one satiety signal that substantially corresponds to at least one neuro-electrical signal that is generated in a body and induces a feeling of fullness in the body, and (iv) transmitting the inverted hunger signal and confounding gastrointestinal signal and satiety signal to the subject.

In a preferred embodiment, the inverted hunger signal has a significant portion that is substantially similar to a negative mirror image of at least one neuro-electrical signal that is generated in a body and induces a sensation of hunger in the body.

In another embodiment of the invention, the method for treating eating disorders includes the steps of (i) generating at least one confounding gastrointestinal signal that is adapted to confound neuro-electrical signals that are generated in the body and induce a sensation of hunger, (ii) generating at least one satiety signal that substantially corresponds to at least one neuro-electrical signal that is generated in a body and induces a feeling of fullness in the body, and (iii) transmitting the confounding gastrointestinal signal and satiety signal to the subject.

In another embodiment of the invention, the method for treating eating disorders includes the steps of (i) capturing at least one neuro-electrical signal that is generated in the body and induces a feeling of fullness in the body, (ii) generating at least one satiety signal based on the neuro-electrical signal, the satiety signal substantially corresponding to the neuro-electrical signal, (iii) generating at least one inverted hunger signal, the inverted hunger signal having at least a portion thereof that is substantially similar to a negative mirror image of a portion of at least one neuro-electrical signal that is generated in a body and induces a sensation of hunger in the body, (iv) sensing food intake in a subject over at least a first period of time, and (v) transmitting the satiety signal and inverted hunger signal to the subject.

In one embodiment, the satiety signal and inverted hunger signal are transmitted to the subject when the food intake of the subject exceeds a predetermined threshold level during the first period of time.

In another embodiment of the invention, the method for treating eating disorders includes the steps of (i) capturing at least one neuro-electrical signal that is generated in the body and induces a feeling of fullness in the body, (ii) generating at least one satiety signal based on the neuro-electrical signal, the satiety signal substantially corresponding to the neuro-electrical signal, (iii) generating at least one inverted hunger signal, the inverted hunger signal having at least a portion thereof that is substantially similar to a negative mirror image of a portion of at least one neuro-electrical signal that is generated in a body and induces a sensation of hunger in the body, (iv) generating at least one confounding gastrointestinal signal, (v) sensing food intake in a subject over at least a first period of time, and (vi) transmitting the satiety signal and inverted hunger signal and confounding gastrointestinal signal to the subject.

In one embodiment, the satiety signal and inverted hunger signal and confounding gastrointestinal signal are transmitted to the subject when the food intake of the subject exceeds a predetermined threshold level during the first period of time.

In another embodiment of the invention, the method for treating eating disorders includes the steps of (i) capturing at least one neuro-electrical signal that is generated in the body and induces a feeling of fullness in the body, (ii) generating at least one satiety signal based on the neuro-electrical signal, the satiety signal substantially corresponding to the neuro-electrical signal, (iii) generating at least one confounding gastrointestinal signal, (iv) sensing food intake in a subject over at least a first period of time, and (v) transmitting the satiety signal and confounding gastrointestinal signal to the subject.

In one embodiment, the satiety signal and confounding gastrointestinal signal are transmitted to the subject when the food intake of the subject exceeds a predetermined threshold level during the first period of time.

In some embodiments of the invention, the method for treating eating disorders also includes the step of capturing at least a second neuro-electrical signal that is generated in the body and induces a sensation of hunger in the body.

In another embodiment of the invention, the method for treating eating disorders includes the steps of (i) generating at least one inverted satiety signal, the inverted satiety signal having at least a portion thereof that is substantially similar to a negative mirror image of a portion of at least one neuro-electrical signal that is generated in a body and induces a feeling of fullness in the body and (ii) transmitting the inverted satiety signal to the subject.

In another embodiment of the invention, the method for treating eating disorders includes the steps of (i) generating at least one inverted satiety signal, the inverted satiety signal having at least a portion thereof that is substantially similar to a negative mirror image of a portion of at least one neuro-electrical signal that is generated in a body and induces a feeling of fullness in the body, (ii) generating at least one hunger signal that substantially corresponds to at least one neuro-electrical signal that is generated in a body and induces a sensation of hunger in the body, and (iii) transmitting the inverted satiety signal and hunger signal to the subject.

In another embodiment of the invention, the method for treating eating disorders includes the steps of (i) generating at least one inverted satiety signal, the inverted satiety signal having at least a portion thereof that is substantially similar to a negative mirror image of a portion of at least one neuro-electrical signal that is generated in a body and induces a feeling of fullness in the body, (ii) generating at least one confounding gastrointestinal signal that is adapted to confound neuro-electrical signals that are generated in the body and induce a feeling of fullness, (iii) generating at least one hunger signal that substantially corresponds to at least one neuro-electrical signal that is generated in a body and induces a sensation of hunger in the body, and (iv) transmitting the inverted satiety signal and confounding gastrointestinal signal and hunger signal to the subject.

In a preferred embodiment, the inverted satiety signal has a significant portion that is substantially similar to a negative mirror image of at least one neuro-electrical signal that is generated in a body and induces a feeling of fullness in the body.

In another embodiment of the invention, the method for treating eating disorders includes the steps of (i) generating at least one confounding gastrointestinal signal that is adapted to confound neuro-electrical signals that are generated in the body and induce a feeling of fullness, (ii) generating at least one hunger signal that substantially corresponds to at least one neuro-electrical signal that is generated in a body and induces a sensation of hunger in the body, and (iii) transmitting the confounding gastrointestinal signal and hunger signal to the subject.

In another embodiment of the invention, the method for treating eating disorders includes the steps of (i) capturing at least one neuro-electrical signal that is generated in the body and induces a sensation of hunger in the body, (ii) generating at least one hunger signal based on the neuro-electrical signal, the hunger signal substantially corresponding to the neuro-electrical signal, (iii) generating at least one inverted satiety signal, the inverted satiety signal having at least a portion thereof that is substantially similar to a negative mirror image of a portion of at least one neuro-electrical signal that is generated in a body and induces a feeling of fullness in the body, (iv) sensing food intake in a subject over at least a first period of time, and (v) transmitting the hunger signal and inverted satiety signal to the subject.

In one embodiment, the hunger signal and inverted satiety signal are transmitted to the subject when the food intake of the subject falls below a predetermined threshold level during the first period of time.

In another embodiment of the invention, the method for treating eating disorders includes the steps of (i) capturing at least one neuro-electrical signal that is generated in the body and induces a sensation of hunger in the body, (ii) generating at least one hunger signal based on the neuro-electrical signal, the hunger signal substantially corresponding to the neuro-electrical signal, (iii) generating at least one inverted satiety signal, the inverted satiety signal having at least a portion thereof that is substantially similar to a negative mirror image of a portion of at least one neuro-electrical signal that is generated in a body and induces a feeling of fullness in the body, (iv) generating at least one confounding gastrointestinal signal, (v) sensing food intake in a subject over at least a first period of time, and (vi) transmitting the hunger signal and inverted satiety signal and confounding gastrointestinal signal to the subject.

In one embodiment, the hunger signal and inverted satiety signal and confounding gastrointestinal signal are transmitted to the subject when the food intake of the subject falls below a predetermined threshold level during the first period of time.

In another embodiment of the invention, the method for treating eating disorders includes the steps of (i) capturing at least one neuro-electrical signal that is generated in the body and induces a sensation of hunger in the body, (ii) generating at least one hunger signal based on the neuro-electrical signal, the hunger signal substantially corresponding to the neuro-electrical signal, (iii) generating at least one confounding gastrointestinal signal, (iv) sensing food intake in a subject over at least a first period of time, and (v) transmitting the hunger signal and confounding gastrointestinal signal to the subject.

In one embodiment, the hunger signal and confounding gastrointestinal signal are transmitted to the subject when the food intake of the subject falls below a predetermined threshold level during the first period of time.

In some embodiments of the invention, the method for treating eating disorders also includes the step of capturing at least a second neuro-electrical signal that is generated in the body and induces a feeling of fullness in the body.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIGS. 1 and 2 are an illustrations of neuro-electrical signals that are generated in the body at a first period of time (i.e. stomach empty) and second period of time (i.e. stomach full);

FIG. 3 is a schematic illustration of one embodiment of a food intake control system, according to the invention;

FIG. 4 is a schematic illustration of another embodiment of a food intake control system, according to the invention;

FIG. 5 is a schematic illustration of another embodiment of a food intake control system, according to the invention;

FIG. 6 is a schematic illustration of yet another embodiment of a food intake control system, according to the invention; and

FIG. 7 is a schematic illustration of one embodiment of a neuro-electrical satiety signal that has been generated by the process means of the invention.

### DETAILED DESCRIPTION OF THE INVENTION

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred systems and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a waveform signal” includes two or more such signals; reference to “a digestive system disorder” includes two or more such disorders and the like.

### Definitions

The terms “patient” and “subject”, as used herein, mean and include humans and animals.

The term “nervous system”, as used herein, means and includes the central nervous system, including the spinal cord, medulla oblongata, pons, cerebellum, midbrain, diencephalon and cerebral hemisphere, and the peripheral nervous system, including the neurons and glia.

The term “plexus”, as used herein, means and includes a branching or tangle of nerve fibers outside the central nervous system.

The term “ganglion”, as used herein, means and includes a group or groups of nerve cell bodies located outside the central nervous system.

The terms “vagus nerve” and “vagus nerve bundle” are used interchangeably herein and mean and include one of the twelve (12) pair of cranial nerves that emanate from the medulla oblongata.

The terms “waveform signal” and “neuro-electrical signal”, as used herein, mean and include a composite electrical signal that is generated in the body and carried by neurons in the body, including neurocodes, neurosignals and components and segments thereof.

The term “gastrointestinal function”, as used herein, means and includes, the operation of all of the organs and structures of the digestive system that are involved in the process of digestion.

The terms “satiety”, “satiety effect”, and “gastrointestinal effect”, as used herein, mean a quality or state associated with food intake, including, without limitation, a feeling of fullness and sensation of hunger.

The term “satiety signal”, as used herein, means an electrical signal that produces or induces a feeling of fullness in a subject when transmitted thereto.

The term “hunger signal”, as used herein, means an electrical signal that produces or induces a sensation of hunger in a subject when transmitted thereto.

The term “gastrointestinal signal”, as used herein, means and includes an electrical signal that produces or induces a gastrointestinal effect in a subject when transmitted thereto, including, without limitation, a sensation of fullness and a sensation of hunger. The term “gastrointestinal signal” thus includes “satiety signals” and “hunger signals”.

The term “inverted satiety signal”, as used herein, means an electrical signal having at least a portion thereof that is substantially similar to a negative mirror image of a portion of a neuro-electrical signal that induces or produces a feeling of fullness in the body. In a preferred embodiment of the invention, a substantial portion of the “inverted satiety signal” is similar to a negative mirror image of a substantial portion of a neuro-electrical signal that induces or produces a feeling of fullness in the body, whereby when an “inverted satiety signal” is transmitted to a subject, the “inverted satiety signal” restricts the transfer of afferent information associated with a naturally generated neuro-electrical signal (i.e. feeling of fullness) to the brain.

The term “inverted hunger signal”, as used herein, means an electrical signal having at least a portion thereof that is substantially similar to a negative mirror image of a portion of a neuro-electrical signal that induces or produces a sensation of hunger in the body. In a preferred embodiment of the invention, a substantial portion of the “inverted hunger signal” is similar to a negative mirror image of a substantial portion of a neuro-electrical signal that induces or produces a sensation of hunger in the body, whereby when an “inverted hunger signal” is transmitted to a subject, the “inverted hunger signal” restricts the transfer of afferent information associated with a naturally generated neuro-electrical signal (i.e. sensation of hunger) to the brain.

The term “confounding gastrointestinal signal”, as used herein, means and includes an electrical signal that is adapted to confound neuro-electrical signals that are generated in the body and induce a gastrointestinal effect in the body.

The term “eating disorder”, as used herein, means and includes, without limitation, compulsive eating and obesity, bulimia and anorexia nervosa.

As discussed in detail in Co-pending U.S. application Ser. No. 11/134,767, which is incorporated by reference herein in its entirety, the vagus nerve bundle, which contains both afferent and efferent pathways, conducts neurosignals from the medulla oblongata to direct aspects of the digestive process, including the secretion of digestive chemicals, operation of the salivary glands and regulation of gastrointestinal muscles (e.g., puborectalis, puboccygeus and iliococcygeus muscles). As indicated above, the vagus nerve bundle also plays a significant role in mediating afferent information from the stomach to the satiety centers of the brain.

Various “electrical stimulation” methods and systems have thus been developed to transmit signals to or stimulate the vagus nerve to produce or induce a satiety effect in the body and, hence, regulate food consumption. The signals do not, however, correspond to short or long-term signals that are naturally generated in the body.

There are thus several disadvantages and drawbacks associated with conventional “electrical stimulation” methods and systems. A significant drawback is that pulses or signals having “high”, and in many instances, excessive signal levels are typically transmitted to a subject, which can, and in many instances will, cause rapid deterioration of the nerve-stimulator connection and/or adverse responses, such as pain, nausea or suppressed and/or irregular heart or respiratory rhythm.

As will be readily apparent to one having ordinary skill in the art, the present invention substantially reduces or eliminates the disadvantages and drawbacks associated with prior art systems and methods for treating eating disorders. As discussed in detail below, the method for treating eating disorders, in accordance with some embodiments of the invention, includes the step of transmitting at least one gastrointestinal signal (i.e., satiety or hunger signal) to a subject that preferably substantially corresponds to or is representative of at least one neuro-electrical signal that is naturally generated in the body and produces a gastrointestinal effect in the body. In one embodiment, the gastrointestinal signal (i.e., satiety signal) produces or induces a feeling of fullness. In another embodiment, the gastrointestinal signal (i.e., hunger signal) produces or induces a sensation of hunger.

In some embodiments of the invention, the methods for treating eating disorders includes the steps of capturing at least one neuro-electrical signal from a body that induces a feeling of fullness in the body and/or at least one neuro-electrical signal from the body that induces a sensation of hunger in the body. More preferably, a plurality of the noted signals is captured from the body.

Suitable neuro-electrical signals that produce a satiety effect in the body can be captured or collected from the vagus nerve bundle. A preferred location is in the neck region of the stomach, which is innervated by the vagus nerve.

According to the invention, the captured neuro-electrical signals are then processed by conventional means to produce satiety and hunger signals that are based on the neuro-electrical signals. Methods and systems for capturing neuro-electrical signals from nerves, and for storing, processing and transmitting neuro-electrical signals are set forth in Co-Pending U.S. patent application Ser. No. 11/125,480, filed May 9, 2005 and Ser. No. 10/000,005, filed Nov. 20, 2001; which are incorporated by reference herein in their entirety.

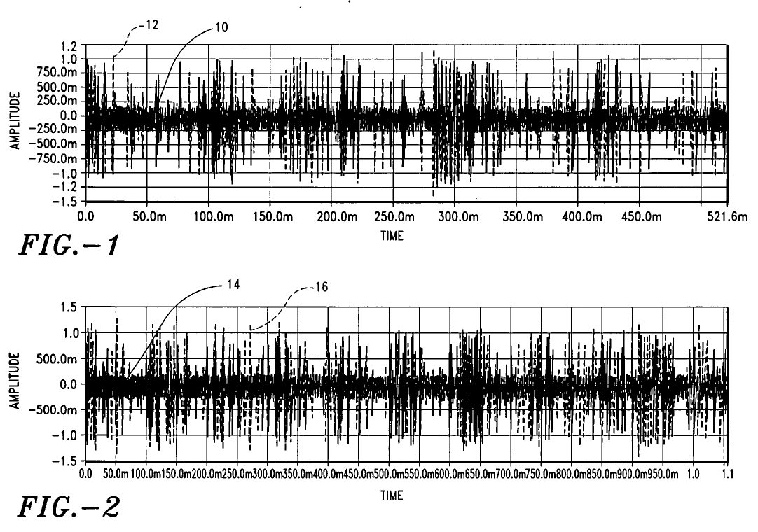

Referring now to FIGS. 1 and 2, there is shown an illustration of neuro-electrical signals 10, 12 that are generated in the body. Referring first to FIG. 1, signal 10 reflects a signal emanating from an empty stomach of a mammal (i.e. pig) at a first period of time. Signal 12 reflects a signal emanating from a full stomach at a second period of time (i.e. ˜10 minutes).

Referring first to FIG. 2, signal 14 similarly reflects a signal emanating from an empty stomach of a mammal at a first period of time. Signal 16 reflects a signal emanating from a full stomach at a second period of time (i.e., ˜10 minutes).

It can be seen from FIGS. 1 and 2 that the neuro-electrical signals include a plurality of “spikes”. Applicants have also found that the signal spikes generally follow a timed sequence.

In some embodiments of the invention, the method for treating eating disorders preferably includes the step of transmitting an inverted satiety signal or inverted hunger signal to a subject. According to the invention, the inverted satiety signal comprises an electrical signal having at least a portion thereof that is substantially similar to a negative mirror image of at least a portion of a neuro-electrical signal that induces or produces a feeling of fullness in the body. In a preferred embodiment of the invention, a substantial portion of the inverted satiety signal is similar to a negative mirror image of a substantial portion of a neuro-electrical signal that induces or produces a feeling of fullness in the body. Thus, when an inverted satiety signal is transmitted to a subject, the inverted satiety signal restricts the transfer of afferent information associated with a naturally generated “target” neuro-electrical signal to the brain, i.e. partially or fully cancels out the target neuro-electrical signal that is inducing a feeling of fullness.

According to the invention, the inverted hunger signal comprises an electrical signal having at least a portion thereof that is substantially similar to a negative mirror image of at least a portion of a neuro-electrical signal that induces or produces a sensation of hunger in the body. In a preferred embodiment of the invention, a substantial portion of the inverted hunger signal is similar to a negative mirror image of a substantial portion of a neuro-electrical signal that induces or produces a sensation of hunger in the body. Thus, when an inverted hunger signal is transmitted to a subject, the inverted hunger signal similarly restricts the transfer of afferent information associated with a naturally generated “target” neuro-electrical signal to the brain, i.e. partially or fully cancels out the target neuro-electrical signal that is inducing a sensation of hunger.

In some embodiments of the invention, the method for treating eating disorders also preferably includes the step of transmitting a confounding gastrointestinal signal to a subject. In a preferred embodiment, the confounding gastrointestinal signal is designed and adapted to confound neuro-electrical signals that are generated in the body and induce a gastrointestinal effect in the body.

According to the invention, the confounding gastrointestinal signal can comprise various forms and can be adapted to modulate or modify naturally generated neuro-electrical signals in numerous ways. In one envisioned embodiment of the invention, the confounding gastrointestinal signal is adapted to substantially abate the effectiveness of the naturally generated neuro-electrical signal when transmitted to a subject. Such a signal is often referred to in the art as a “keep-busy” signal.

By way of example, in the case of a compulsive eating event by a subject, when a confounding gastrointestinal signal is transmitted to the subject the confounding gastrointestinal signal would abate the effectiveness of the neuro-electrical signal (or signals) that is inducing a sensation of hunger.

In another envisioned embodiment of the invention, the confounding gastrointestinal signal is adapted to modify the naturally generated neuro-electrical signal, e.g., amplitude of the signal, duration of signal, etc.

One suitable confounding signal is disclosed in Co-pending U.S. application Ser. No. 11/393,194; which is incorporated herein in its entirety.

According to one embodiment of the invention, when an inverted hunger signal and a satiety signal are transmitted to the subject the inverted hunger signal abates the effectiveness of the neuro-electrical signal (or signals) that is inducing a sensation of hunger, while the satiety signal induces a desired gastrointestinal effect; in this instance, a feeling of fullness.

Similarly, when a confounding gastrointestinal signal and a satiety signal are transmitted to the subject the confounding gastrointestinal signal abates the effectiveness of the neuro-electrical signal (or signals) that is inducing a sensation of hunger, while the satiety signal induces a feeling of fullness.

According to another embodiment of the invention, when an inverted satiety signal and a hunger signal are transmitted to the subject the inverted satiety signal abates the effectiveness of the neuro-electrical signal (or signals) that is inducing a feeling of fullness, while the hunger signal induces a desired gastrointestinal effect; in this instance, a sensation of hunger.

Similarly, when a confounding gastrointestinal signal and a hunger signal are transmitted to the subject the confounding gastrointestinal signal abates the effectiveness of the neuro-electrical signal (or signals) that is inducing a feeling of fullness, while the hunger signal similarly induces a sensation of hunger.

In some embodiments, the method for treating eating disorders also includes the step of monitoring the subject's food intake, i.e. the quantity of food consumed. One suitable means for monitoring or ascertaining food intake comprises implanting one or more sensing electrodes in or at the esophagus to detect the passage of food as the subject swallows. The swallows are then summed over a predetermined time interval to estimate the amount of food consumed in that interval. According to the invention, a generated neuro-electrical satiety signal can then be transmitted to the subject if the estimated food consumption exceeds a predetermined threshold level.

Since the caloric intake of similar volumes (or quantities) of two different foods can be significantly different, in one envisioned embodiment of the invention, the method of monitoring (or ascertaining) a subject's food intake includes ascertaining the approximate caloric intake. One suitable means of ascertaining the calories associated with a quantity of selected foods is to include a table of foods and associated calories or, more preferably, calories per weight or volume, in the control system module or processor (which are described below).

The subject would then input the meal (or desired food) that is about to be consumed into the system and the system would determine the caloric value associated with each inputted food. Based on a pre-programmed caloric intake, or more preferably, a caloric intake over a predetermined period of time, which is tailored to the subject, the system would determine a target, desired range of food intake for the inputted food(s).

Alternatively, the target calories and, hence, volume of food intake can be determined from various nutritional formulae or a standardized caloric table. By way of example, referring to Table I, there is shown a table of estimated amounts of calories needed to maintain energy balance for various gender and age groups at three different levels of physical activity. The noted levels are based on Estimated Energy Requirements (EER) from the Institute of Medicine Dietary Reference Intakes macronutrients report, 2002; calculated by gender, age, and activity level for reference-sized individuals.

“Reference size”, as determined by IOM, is based on median height and weight for ages up to age 18 years of age and median height and weight for that height to give a BMI of 21.5 for adult females and 22.5 for adult males.

TABLE I

| Gender | Age (yrs) | Sedentary^{a} | Moderately Active^{b} | Active^{c} |
|---|---|---|---|---|
| Child | 2-3 | 1,000 | 1,000-1,400 | 1,000-1,400 |
| Female | 4-8 | 1,200 | 1,400-1,600 | 1,400-1,800 |
| | 9-13 | 1,600 | 1,600-2,000 | 1,800-2,200 |
| | 14-18 | 1,800 | 2,000 | 2,400 |
| | 19-30 | 2,000 | 2,000-2,200 | 2,400 |
| | 31-50 | 1,800 | 2,000 | 2,200 |
| | 51+ | 1,600 | 1,800 | 2,000-2,200 |
| Male | 4-8 | 1,400 | 1,400-1,600 | 1,600-2,000 |
| | 9-13 | 1,800 | 1,800-2,200 | 2,000-2,600 |
| | 14-18 | 2,200 | 2,400-2,800 | 2,800-3,200 |
| | 19-30 | 2,400 | 2,600-2,800 | 3,000 |
| | 31-50 | 2,200 | 2,400-2,600 | 2,800-3,000 |
| | 51+ | 2,000 | 2,200-2,400 | 2,400-2,800 |

^{a}Sedentary means a lifestyle that includes only the light physical activity associated with typical day-to-day life.
^{b}Moderately active means a lifestyle that includes physical activity equivalent to walking about 1.5 to 3 miles per day at 3 to 4 miles per hour, in addition to the light physical activity associated with typical day-to-day life.
^{c}Active means a lifestyle that includes physical activity equivalent to walking more than 3 miles per day at 3 to 4 miles per hour, in addition to the light physical activity associated with typical day-to-day life.

According to the invention, the caloric intake and, hence, quantity of food (i.e., food intake) can be adjusted upward or downward to induce weight loss or weight gain.

Thus, a method for treating eating disorders, in accordance with one embodiment of the invention, includes the steps of (i) generating a confounding gastrointestinal signal, (ii) generating a gastrointestinal signal that substantially corresponds to a neuro-electrical signal that is generated in a body and produces a gastrointestinal effect in the body, (iii) sensing food intake in a subject over at least a first period of time, and (iv) transmitting the confounding gastrointestinal signal and gastrointestinal signal to the subject.

In one embodiment of the invention, the gastrointestinal effect comprises a feeling of fullness and the gastrointestinal signal is transmitted to the subject if the food intake “exceeds” a predetermined threshold level during the first period of time. According to the invention, when the gastrointestinal signal (i.e. satiety signal) is transmitted to the subject, the subject experiences a satisfied feeling of fullness at a predetermined level of food consumption that is sufficient to maintain physiologic needs, but supportive of weight reduction. The noted method of the invention can thus be effectively employed to treat obesity and control excessive overeating. A similar method can also be employed to treat bulimia.

In another embodiment, the gastrointestinal effect comprises a sensation of hunger and the gastrointestinal signal is transmitted to the subject if the food intake “falls below” a predetermined threshold level during the first period of time. According to the invention, when the gastrointestinal signal (i.e. hunger signal) is transmitted to the subject, the subject experiences a sensation of hunger and, hence, is urged to eat. The noted method can thus be effectively employed to treat anorexia nervosa. The method can also be employed to modify or control food consumption after various surgical procedures.

In alternative embodiments of the invention, the methods include the pre-programmed or timed transmission of a gastrointestinal (i.e., satiety or hunger) signal, inverted satiety signal, inverted hunger signal or confounding gastrointestinal signal. For example, in the case of an obese or bulimic subject, a satiety signal and a confounding gastrointestinal signal and/or an inverted hunger signal can be transmitted at set intervals at, near and/or between customary meal times to induce a feeling of fullness. In the case of an anorexic subject, a hunger signal and a confounding gastrointestinal signal and/or an inverted satiety signal can be transmitted at prescribed meal times to induce a sensation of hunger.

As discussed in detail herein, alternatively, or in addition with sensing food intake and transmitting a gastrointestinal signal or confounding gastrointestinal signal or inverted satiety or hunger signal in response thereto and/or timed transmission of a gastrointestinal signal or confounding gastrointestinal signal or inverted satiety or hunger signal, the transmission of the gastrointestinal signals, confounding gastrointestinal signals and inverted satiety or hunger signals can also be accomplished manually. As will be appreciated by one having skill in the art, manual transmission of a gastrointestinal signal and/or confounding gastrointestinal signal and/or inverted satiety or hunger signal is useful in situations where the subject has an earnest desire to control his or her eating behavior, but requires supportive measures due to insufficient will power to refrain from compulsive and/or damaging behavior.

As indicated, in some embodiments of the invention, the method for treating eating disorders includes the steps of capturing at least one neuro-electrical signal from a body that induces a feeling of fullness in the body and/or at least one neuro-electrical signal from the body that induces a sensation of hunger in the body. According to one embodiment of the invention, the captured neuro-electrical signals are preferably transmitted to a processor or control module. Preferably, the control module includes storage means adapted to store the captured signals.

According to the invention, the captured neuro-electrical signals can be processed by known means to generate a gastrointestinal signal (i.e., satiety or hunger) that similarly produces a gastrointestinal effect in the body, and substantially corresponds to or is representative of at least one captured neuro-electrical signal. The generated gastrointestinal signal is similarly preferably stored in the storage means of the control module.

The captured neuro-electrical signals can also be processed by known means to generate the inverted satiety and hunger signals of the invention.

In response to a pre-programmed event, e.g., food intake exceeding a predetermined threshold level, food intake falling below a predetermined threshold level, a pre-programmed period of time or time interval or manual activation, the desired signal (or signals), i.e. generated gastrointestinal signal and/or confounding gastrointestinal signal and/or inverted satiety or hunger signal, is accessed from the storage means and transmitted to the subject via a transmitter (or probe).

According to the invention, the applied voltage of gastrointestinal signals, confounding gastrointestinal signals, inverted satiety signals and inverted hunger signals can be up to 20 volts to allow for voltage loss during the transmission of the signals. Preferably, current is maintained to less than 2 amp output.

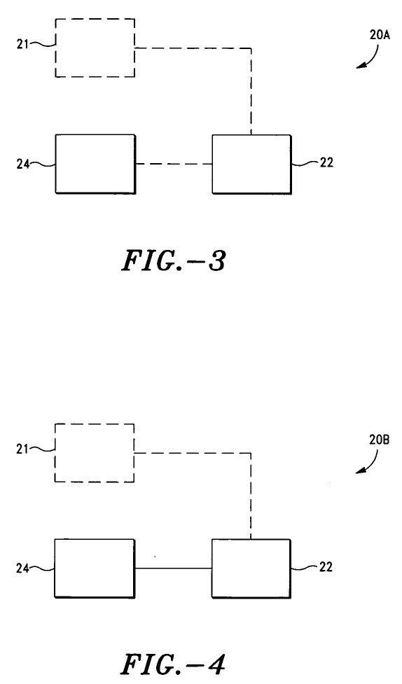

Referring now to FIG. 3, there is shown a schematic illustration of one embodiment of a food intake control system 20A of the invention. As illustrated in FIG. 3, the control system 20A includes a control module 22, which is adapted to receive neuro-electrical signals from a signal sensor (shown in phantom and designated 21) that is in communication with a subject, and at least one treatment member 24.

The control module 22 is further adapted to generate inverted satiety and hunger signals, confounding gastrointestinal signals and gastrointestinal signals that substantially correspond to or are representative of neuro-electrical signals that are generated in the body and produce a gastrointestinal effect in the body, and transmit the inverted satiety and hunger signals and/or gastrointestinal signals and/or confounding gastrointestinal signals to the treatment member 24 at predetermined periods of time (or time intervals). The control module is also adapted to transmit the inverted satiety and hunger signals and/or gastrointestinal signals and/or confounding gastrointestinal signals to the treatment member 24 manually, i.e. upon activation of a manual switch (not shown).

The treatment member 24 is adapted to communicate with the body and receives the inverted satiety and hunger signals and/or gastrointestinal signals and/or confounding gastrointestinal signals from the control module 22. According to the invention, the treatment member 24 can comprise an electrode, antenna, a seismic transducer, or any other suitable form of conduction attachment for transmitting the gastrointestinal signals and confounding gastrointestinal signals to a subject.

According to the invention, the treatment member 24 can be attached to appropriate nerves via a surgical process. Such surgery can, for example, be accomplished through a “key-hole” entrance in an endoscopic procedure. If necessary, a more invasive procedure can be employed for more proper placement of the treatment member 24.

Examples of suitable transmission points for transmittal of inverted satiety and hunger signals, gastrointestinal signals and confounding gastrointestinal signals by the treatment member 24 include the neck of the stomach and/or left or right branches of the vagus nerve that is located in the neck.

As illustrated in FIG. 3, the control module 22 and treatment member 24 can be entirely separate elements, which allow system 20A to be operated remotely. According to the invention, the control module 22 can be unique, i.e., tailored to a specific operation and/or subject or can comprise a conventional device.

Referring now to FIG. 4, there is shown a further embodiment of a control system 20B of the invention. As illustrated in FIG. 4, the system 20B is similar to system 20A shown in FIG. 3. However, in this embodiment, the control module 22 and treatment member 24 are connected.

Referring now to FIG. 5, there is shown yet another embodiment of a control system 20C of the invention. As illustrated in FIG. 5, the control system 20C similarly includes a control module 22 and a treatment member 24. The system 20C further includes at least one signal sensor 21.

The system 20C also includes a processing module (or computer) 26. According to the invention, the processing module 26 can be a separate component or a sub-system of a control module 22′, as shown in phantom.

As indicated above, the processing module (or control module) 26 preferably includes storage means adapted to store the captured neuro-electrical signals that produce a gastrointestinal effect in the body. In a preferred embodiment, the processing module 26 is further adapted to extract and store the components of the captured neuro-electrical signals in the storage means according to the function performed by the signal components.

The processing module (or control module) 26 is also preferably adapted to store the generated inverted satiety and hunger signals and confounding gastrointestinal signals of the invention.

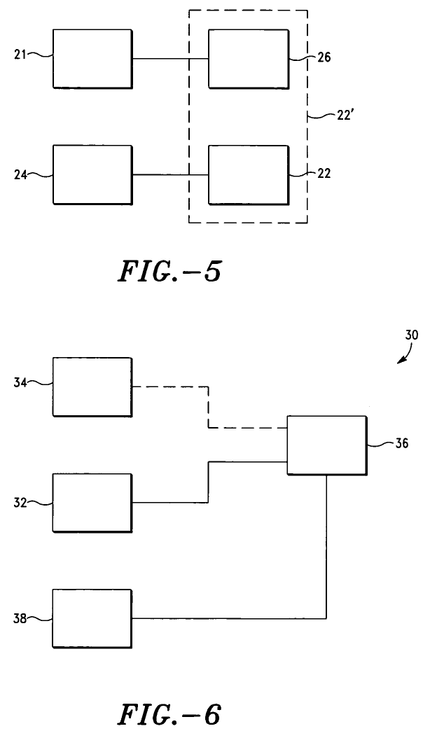

Referring now to FIG. 6, there is shown yet another embodiment of a food intake control system 30. As illustrated in FIG. 6, the system 30 includes at least one food intake sensor 32 that is adapted to monitor the food intake or consumption of a subject and generate at least one signal indicative of the food intake, i.e. food intake signal.

As one having ordinary skill in the art will appreciate, various sensing methods and sensors can be employed within the scope of the invention to monitor food intake. In one embodiment, the method for monitoring food intake comprises implanting one or more sensing electrodes in or at the esophagus to detect the passage of food as the subject swallows. The swallows are then summed over a predetermined time interval to estimate the amount of food consumed in that interval.

According to the invention, motion and pressure sensors, and other physiological devices, such as gastrointestinal bands that are adapted to sense pressure within a gastrointestinal tract structure or pressure changes caused by expansion or contraction of a gastrointestinal tract structure can also be employed.

The system 30 further includes a processor 36, which is adapted to receive the food intake signals from the food intake sensor 32. The processor 36 is further adapted to receive neuro-electrical signals recorded by a signal sensor (shown in phantom and designated 34).

In a preferred embodiment of the invention, the processor 36 includes storage means for storing the captured neuro-electrical signals and food intake signals (and generated inverted satiety and hunger signals, gastrointestinal signals and confounding gastrointestinal signals). The processor 36 is further adapted to extract the components of the neuro-electrical signals and store the signal components in the storage means.

In a preferred embodiment, the processor 36 is programmed to (i) detect when food intake signals reflect that the subject has exceeded a predetermined threshold of food intake in a predetermined period of time or has not consumed sufficient food over a predetermined period of time, (ii) generate a confounding gastrointestinal signal and/or an inverted satiety or hunger signal, and (iii) generate at least one gastrointestinal signal that substantially corresponds to a neuro-electrical signal that is generated in the body and produces a gastrointestinal effect in the body. The processor 36 is preferably further adapted to transmit inverted satiety or hunger signals and/or confounding gastrointestinal signals and/or gastrointestinal signals to the subject in response to a food intake signal reflecting that the subject has exceeded a predetermined threshold of food intake in a predetermined period of time, a food intake signal reflecting that the subject has not consumed a predetermined threshold of food intake in a predetermined period of time, at a predetermined period of time (or time interval) and/or manually, i.e. upon activation of a first manual switch (not shown).

Referring to FIG. 6, the inverted satiety or hunger signals and/or gastrointestinal signals and/or confounding gastrointestinal signals are routed to a transmitter 38 that is adapted to be in communication with the subject's body. The transmitter 38 is adapted to transmit the inverted satiety or hunger signals and/or gastrointestinal signals and/or confounding gastrointestinal signals to the subject (in a similar manner as described above) to regulate the subject's food intake.

In accordance with one embodiment of the invention, the method for treating eating disorders includes the steps of (i) generating at least one inverted hunger signal, the inverted hunger signal having at least a portion thereof that is substantially similar to a negative mirror image of a portion of at least one neuro-electrical signal that is generated in a body and induces a sensation of hunger in the body and (ii) transmitting the inverted hunger signal to the subject.

In a preferred embodiment, the inverted hunger signal has a significant portion that is substantially similar to a negative mirror image of a substantial portion of at least one neuro-electrical signal that is generated in a body and induces a sensation of hunger in the body.

In one embodiment, the inverted hunger signal is transmitted at predetermined time intervals.

In one embodiment, the inverted hunger signal is transmitted manually.

In another embodiment, the inverted hunger signal is transmitted at predetermined time intervals and manually.

In another embodiment of the invention, the method for treating eating disorders includes the steps of (i) generating at least one inverted hunger signal, the inverted hunger signal having at least a portion thereof that is substantially similar to a negative mirror image of a portion of at least one neuro-electrical signal that is generated in a body and induces a sensation of hunger in the body, (ii) generating at least one satiety signal that substantially corresponds to at least one neuro-electrical signal that is generated in a body and induces a feeling of fullness in the body, and (iii) transmitting the inverted hunger signal and satiety signal to the subject.

In one embodiment, the inverted hunger signal is transmitted at first predetermined time intervals.

In one embodiment, the inverted hunger signal is transmitted manually.

In one embodiment, the satiety signal is transmitted at second predetermined time intervals.

In one embodiment, the satiety signal is transmitted manually.

In another embodiment, the inverted hunger signal and satiety signal are transmitted substantially simultaneously at the first predetermined time intervals.

In one embodiment, the inverted hunger signal and satiety signal are transmitted manually.

In another embodiment, the inverted hunger signal and satiety signal are transmitted manually and at the first predetermined time intervals.

In another embodiment of the invention, the method for treating eating disorders includes the steps of (i) generating at least one inverted hunger signal, the inverted hunger signal having at least a portion thereof that is substantially similar to a negative mirror image of a portion of at least one neuro-electrical signal that is generated in a body and induces a sensation of hunger in the body, (ii) generating at least one confounding gastrointestinal signal that is adapted to confound neuro-electrical signals that are generated in the body and induce a sensation of hunger, (iii) generating at least one satiety signal that substantially corresponds to at least one neuro-electrical signal that is generated in a body and induces a feeling of fullness in the body, and (iv) transmitting the inverted hunger signal and confounding gastrointestinal signal and satiety signal to the subject.

In one embodiment, the inverted hunger signal is transmitted at first predetermined time intervals.

In one embodiment, the inverted hunger signal is transmitted manually.

In one embodiment, the confounding gastrointestinal signal is transmitted at second predetermined time intervals.

In one embodiment, the confounding gastrointestinal signal is transmitted manually.

In one embodiment, the satiety signal is transmitted at third predetermined time intervals.

In one embodiment, the satiety signal is transmitted manually.

In another embodiment, the inverted hunger signal and satiety signal are transmitted manually and at the first predetermined time intervals.

In another embodiment, the inverted hunger signal and confounding gastrointestinal signal and satiety signal are transmitted substantially simultaneously at the first predetermined time intervals.

In one embodiment, the inverted hunger signal and confounding gastrointestinal signal and satiety signal are transmitted manually.

In another embodiment, the inverted hunger signal and confounding gastrointestinal signal and satiety signal are transmitted manually and at the first predetermined time intervals.

In another embodiment of the invention, the method for treating eating disorders includes the steps of (i) capturing at least one neuro-electrical signal that is generated in the body and induces a feeling of fullness in the body, (ii) generating at least one satiety signal based on the neuro-electrical signal, the satiety signal substantially corresponding to the neuro-electrical signal, (iii) generating at least one inverted hunger signal, the inverted hunger signal having at least a portion thereof that is substantially similar to a negative mirror image of a portion of at least one neuro-electrical signal that is generated in a body and induces a sensation of hunger in the body, (iv) sensing food intake in a subject over at least a first period of time, and (v) transmitting the satiety signal and inverted hunger signal to the subject.

In one embodiment, the inverted hunger signal is transmitted when the food intake of the subject exceeds a predetermined threshold level during the first period of time.

In one embodiment, the inverted hunger signal is transmitted at first predetermined time intervals.

In one embodiment, the inverted hunger signal is transmitted manually.

In one embodiment, the satiety signal is transmitted when the food intake of the subject exceeds a predetermined threshold level during the first period of time.

In one embodiment, the satiety signal is transmitted at second predetermined time intervals.

In one embodiment, the satiety signal is transmitted manually.

In one embodiment, the inverted hunger signal and satiety signal are transmitted when the food intake of the subject exceeds a predetermined threshold level during the first period of time.

In another embodiment, the inverted hunger signal and satiety signal are transmitted substantially simultaneously at the first predetermined time intervals.

In one embodiment, the inverted hunger signal and satiety signal are transmitted manually.

In another embodiment, the inverted hunger signal and satiety signal are transmitted manually and at predetermined time intervals.

In another embodiment, the inverted hunger signal and satiety signal are transmitted when the food intake of the subject exceeds a predetermined threshold level during the first period of time and manually and at the first predetermined time intervals.

In another embodiment of the invention, the method for treating eating disorders includes the steps of (i) capturing at least one neuro-electrical signal that is generated in the body and induces a feeling of fullness in the body, (ii) generating at least one satiety signal based on the neuro-electrical signal, the satiety signal substantially corresponding to the neuro-electrical signal, (iii) generating at least one inverted hunger signal, the inverted hunger signal having at least a portion thereof that is substantially similar to a negative mirror image of a portion of at least one neuro-electrical signal that is generated in a body and induces a sensation of hunger in the body, (iv) generating at least one confounding gastrointestinal signal, (v) sensing food intake in a subject over at least a first period of time, and (vi) transmitting the satiety signal and inverted hunger signal and confounding gastrointestinal signal to the subject.

In one embodiment, the inverted hunger signal is transmitted when the food intake of the subject exceeds a predetermined threshold level during the first period of time.

In one embodiment, the inverted hunger signal is transmitted at first predetermined time intervals.

In one embodiment, the inverted hunger signal is transmitted manually.

In one embodiment, the satiety signal is transmitted when the food intake of the subject exceeds a predetermined threshold level during the first period of time.

In one embodiment, the satiety signal is transmitted at second predetermined time intervals.

In one embodiment, the satiety signal is transmitted manually.

In one embodiment, the confounding gastrointestinal signal is transmitted when the food intake of the subject exceeds a predetermined threshold level during the first period of time.

In one embodiment, the confounding gastrointestinal signal is transmitted at first predetermined time intervals.

In one embodiment, the confounding gastrointestinal signal is transmitted manually.

In one embodiment, the inverted hunger signal and satiety signal are transmitted when the food intake of the subject exceeds a predetermined threshold level during the first period of time.

In another embodiment, the inverted hunger signal and satiety signal are transmitted substantially simultaneously at the first predetermined time intervals.

In one embodiment, the inverted hunger signal and satiety signal are transmitted manually.

In another embodiment, the inverted hunger signal and satiety signal are transmitted manually and at predetermined time intervals.

In another embodiment, the inverted hunger signal and confounding gastrointestinal signal and satiety signal are transmitted when the food intake of the subject exceeds a predetermined threshold level during the first period of time and manually and at the first predetermined time intervals.

In one embodiment, the inverted hunger signal and confounding gastrointestinal signal and satiety signal are transmitted when the food intake of the subject exceeds a predetermined threshold level during the first period of time.

In another embodiment, the inverted hunger signal and confounding gastrointestinal signal and satiety signal are transmitted substantially simultaneously at the first predetermined time intervals.

In one embodiment, the inverted hunger signal and confounding gastrointestinal signal and satiety signal are transmitted manually.

In another embodiment, the inverted hunger signal and confounding gastrointestinal signal and satiety signal are transmitted manually and at predetermined time intervals.

In another embodiment, the inverted hunger signal and confounding gastrointestinal signal and satiety signal are transmitted when the food intake of the subject exceeds a predetermined threshold level during the first period of time and manually and at the first predetermined time intervals.

In another embodiment of the invention, the method for treating eating disorders includes the steps of (i) capturing at least one neuro-electrical signal that is generated in the body and induces a feeling of fullness in the body, (ii) generating at least one satiety signal based on the neuro-electrical signal, the satiety signal substantially corresponding to the neuro-electrical signal, (iii) generating at least one confounding gastrointestinal signal, (iv) sensing food intake in a subject over at least a first period of time, and (v) transmitting the satiety signal and confounding gastrointestinal signal to the subject when the food intake of the subject exceeds a predetermined threshold level during the first period of time.

In some embodiments of the invention, the noted methods for treating eating disorders also include the step of capturing at least a second neuro-electrical signal that is generated in the body and induces a sensation of hunger in the body.

In another embodiment of the invention, the method for treating eating disorders includes the steps of (i) generating at least one confounding gastrointestinal signal that is adapted to confound neuro-electrical signals that are generated in the body and induce a sensation of hunger, (ii) generating at least one satiety signal that substantially corresponds to at least one neuro-electrical signal that is generated in a body and induces a feeling of fullness in the body, and (iii) transmitting the confounding gastrointestinal signal and satiety signal to the subject.

In one embodiment, the satiety signal is transmitted at first predetermined time intervals.

In one embodiment, the satiety signal is transmitted manually.

In one embodiment, the confounding gastrointestinal signal is transmitted at second predetermined time intervals.

In one embodiment, the confounding gastrointestinal signal is transmitted manually.

In another embodiment, the satiety signal and confounding gastrointestinal signal are transmitted substantially simultaneously at the first predetermined time intervals.

In one embodiment, the satiety signal and confounding gastrointestinal signal are transmitted manually.

In another embodiment, the satiety signal and confounding gastrointestinal signal are transmitted manually and at predetermined time intervals.

In accordance with one embodiment of the invention, the method for treating eating disorders includes the steps of (i) generating at least one inverted satiety signal, the inverted satiety signal having at least a portion thereof that is substantially similar to a negative mirror image of a portion of at least one neuro-electrical signal that is generated in a body and induces a feeling of fullness in the body and (ii) transmitting the inverted satiety signal to the subject.

In a preferred embodiment, the inverted satiety signal has a significant portion that is substantially similar to a negative mirror image of a substantial portion of at least one neuro-electrical signal that is generated in a body and induces a feeling of fullness in the body.

In one embodiment, the inverted satiety signal is transmitted at predetermined time intervals.

In one embodiment, the inverted satiety signal is transmitted manually.

In another embodiment, the inverted satiety signal is transmitted at predetermined time intervals and manually.

In another embodiment of the invention, the method for treating eating disorders includes the steps of (i) generating at least one inverted satiety signal, the inverted satiety signal having at least a portion thereof that is substantially similar to a negative mirror image of a portion of at least one neuro-electrical signal that is generated in a body and induces a feeling of fullness in the body, (ii) generating at least one hunger signal that substantially corresponds to at least one neuro-electrical signal that is generated in a body and induces a sensation of hunger in the body, and (iii) transmitting the inverted satiety signal and hunger signal to the subject.

In one embodiment, the inverted satiety signal is transmitted at first predetermined time intervals.

In one embodiment, the inverted satiety signal is transmitted manually.

In one embodiment, the hunger signal is transmitted at second predetermined time intervals.

In one embodiment, the hunger signal is transmitted manually.

In another embodiment, the inverted satiety signal and hunger signal are transmitted substantially simultaneously at the first predetermined time intervals.

In one embodiment, the inverted satiety signal and hunger signal are transmitted manually.

In another embodiment, the inverted satiety signal and hunger signal are transmitted manually and at the first predetermined time intervals.

In another embodiment of the invention, the method for treating eating disorders includes the steps of (i) generating at least one inverted satiety signal, the inverted satiety signal having at least a portion thereof that is substantially similar to a negative mirror image of a portion of at least one neuro-electrical signal that is generated in a body and induces a feeling of fullness in the body, (ii) generating at least one confounding gastrointestinal signal that is adapted to confound neuro-electrical signals that are generated in the body and induce a feeling of fullness, (iii) generating at least one hunger signal that substantially corresponds to at least one neuro-electrical signal that is generated in a body and induces a sensation of hunger in the body, and (iv) transmitting the inverted satiety signal and confounding gastrointestinal signal and hunger signal to the subject.

In one embodiment, the inverted satiety signal is transmitted at first predetermined time intervals.

In one embodiment, the inverted satiety signal is transmitted manually.

In one embodiment, the confounding gastrointestinal signal is transmitted at second predetermined time intervals.

In one embodiment, the confounding gastrointestinal signal is transmitted manually.

In one embodiment, the hunger signal is transmitted at third predetermined time intervals.

In one embodiment, the hunger signal is transmitted manually.

In another embodiment, the inverted satiety signal and hunger signal are transmitted manually and at the first predetermined time intervals.

In another embodiment, the inverted satiety signal and confounding gastrointestinal signal and hunger signal are transmitted substantially simultaneously at the first predetermined time intervals.

In one embodiment, the inverted satiety signal and confounding gastrointestinal signal and hunger signal are transmitted manually.

In another embodiment, the inverted satiety signal and confounding gastrointestinal signal and hunger signal are transmitted manually and at the first predetermined time intervals.

In another embodiment of the invention, the method for treating eating disorders includes the steps of (i) capturing at least one neuro-electrical signal that is generated in the body and induces a sensation of hunger in the body, (ii) generating at least one hunger signal based on the neuro-electrical signal, the hunger signal substantially corresponding to the neuro-electrical signal, (iii) generating at least one inverted satiety signal, the inverted satiety signal having at least a portion thereof that is substantially similar to a negative mirror image of a portion of at least one neuro-electrical signal that is generated in a body and induces a feeling of fullness in the body, (iv) sensing food intake in a subject over at least a first period of time, and (v) transmitting the hunger signal and inverted satiety signal to the subject.

In one embodiment, the inverted satiety signal is transmitted when the food intake of the subject falls below a predetermined threshold level during the first period of time.

In one embodiment, the inverted satiety signal is transmitted at first predetermined time intervals.

In one embodiment, the inverted satiety signal is transmitted manually.

In one embodiment, the hunger signal is transmitted when the food intake of the subject falls below a predetermined threshold level during the first period of time.

In one embodiment, the hunger signal is transmitted at second predetermined time intervals.

In one embodiment, the hunger signal is transmitted manually.

In one embodiment, the inverted satiety signal and hunger signal are transmitted when the food intake of the subject falls below a predetermined threshold level during the first period of time.

In another embodiment, the inverted satiety signal and hunger signal are transmitted substantially simultaneously at the first predetermined time intervals.

In one embodiment, the inverted satiety signal and hunger signal are transmitted manually.

In another embodiment, the inverted satiety signal and hunger signal are transmitted manually and at predetermined time intervals.

In another embodiment, the inverted satiety signal and hunger signal are transmitted when the food intake of the subject falls below a predetermined threshold level during the first period of time and manually and at the first predetermined time intervals.

In another embodiment of the invention, the method for treating eating disorders includes the steps of (i) capturing at least one neuro-electrical signal that is generated in the body and induces a sensation of hunger in the body, (ii) generating at least one hunger signal based on the neuro-electrical signal, the hunger signal substantially corresponding to the neuro-electrical signal, (iii) generating at least one inverted satiety signal, the inverted satiety signal having at least a portion thereof that is substantially similar to a negative mirror image of a portion of at least one neuro-electrical signal that is generated in a body and induces a feeling of fullness in the body, (iv) generating at least one confounding gastrointestinal signal, (v) sensing food intake in a subject over at least a first period of time, and (vi) transmitting the hunger signal and inverted satiety signal and confounding gastrointestinal signal to the subject.

In one embodiment, the inverted satiety signal is transmitted when the food intake of the subject falls below a predetermined threshold level during the first period of time.

In one embodiment, the inverted satiety signal is transmitted at first predetermined time intervals.

In one embodiment, the inverted satiety signal is transmitted manually.

In one embodiment, the hunger signal is transmitted when the food intake of the subject falls below a predetermined threshold level during the first period of time.

In one embodiment, the hunger signal is transmitted at second predetermined time intervals.

In one embodiment, the hunger signal is transmitted manually.

In one embodiment, the confounding gastrointestinal signal is transmitted when the food intake of the subject falls below a predetermined threshold level during the first period of time.

In one embodiment, the confounding gastrointestinal signal is transmitted at first predetermined time intervals.

In one embodiment, the confounding gastrointestinal signal is transmitted manually.

In one embodiment, the inverted satiety signal and hunger signal are transmitted when the food intake of the subject falls below a predetermined threshold level during the first period of time.

In another embodiment, the inverted satiety signal and hunger signal are transmitted substantially simultaneously at the first predetermined time intervals.

In one embodiment, the inverted satiety signal and hunger signal are transmitted manually.

In another embodiment, the inverted satiety signal and hunger signal are transmitted manually and at predetermined time intervals.

In another embodiment, the inverted satiety signal and confounding gastrointestinal signal and hunger signal are transmitted when the food intake of the subject falls below a predetermined threshold level during the first period of time and manually and at the first predetermined time intervals.

In one embodiment, the inverted satiety signal and confounding gastrointestinal signal and hunger signal are transmitted when the food intake of the subject falls below a predetermined threshold level during the first period of time.

In another embodiment, the inverted satiety signal and confounding gastrointestinal signal and hunger signal are transmitted substantially simultaneously at the first predetermined time intervals.

In one embodiment, the inverted satiety signal and confounding gastrointestinal signal and hunger signal are transmitted manually.

In another embodiment, the inverted satiety signal and confounding gastrointestinal signal and hunger signal are transmitted manually and at predetermined time intervals.

In another embodiment, the inverted satiety signal and confounding gastrointestinal signal and hunger signal are transmitted when the food intake of the subject falls below a predetermined threshold level during the first period of time and manually and at the first predetermined time intervals.

In another embodiment of the invention, the method for treating eating disorders includes the steps of (i) capturing at least one neuro-electrical signal that is generated in the body and induces a sensation of hunger in the body, (ii) generating at least one hunger signal based on the neuro-electrical signal, the hunger signal substantially corresponding to the neuro-electrical signal, (iii) generating at least one confounding gastrointestinal signal, (iv) sensing food intake in a subject over at least a first period of time, and (v) transmitting the hunger signal and confounding gastrointestinal signal to the subject when the food intake of the subject falls below a predetermined threshold level during the first period of time.

In some embodiments of the invention, the noted methods for treating eating disorders also include the step of capturing at least a second neuro-electrical signal that is generated in the body and induces a feeling of fullness in the body.

In another embodiment of the invention, the method for treating eating disorders includes the steps of (i) generating at least one confounding gastrointestinal signal that is adapted to confound neuro-electrical signals that are generated in the body and induce a feeling of fullness, (ii) generating at least one hunger signal that substantially corresponds to at least one neuro-electrical signal that is generated in a body and induces a sensation of hunger in the body, and (iii) transmitting the confounding gastrointestinal signal and hunger signal to the subject.

In one embodiment, the hunger signal is transmitted at first predetermined time intervals.

In one embodiment, the hunger signal is transmitted manually.

In one embodiment, the confounding gastrointestinal signal is transmitted at second predetermined time intervals.

In one embodiment, the confounding gastrointestinal signal is transmitted manually.

In another embodiment, the hunger signal and confounding gastrointestinal signal are transmitted substantially simultaneously at the first predetermined time intervals.

In one embodiment, the hunger signal and confounding gastrointestinal signal are transmitted manually.

In another embodiment, the hunger signal and confounding gastrointestinal signal are transmitted manually and at predetermined time intervals.

In each of the noted embodiments of the invention, the generated gastrointestinal signals and inverted satiety and hunger signals and confounding gastrointestinal signals are transmitted to the subject's nervous system.

More preferably, the generated gastrointestinal signals and inverted satiety and hunger signals confounding gastrointestinal signals are transmitted to the vagus nerve.

In one embodiment of the invention, the step of transmitting a gastrointestinal (i.e. satiety and hunger) signal and/or confounding gastrointestinal (i.e. satiety and hunger) signal and/or inverted satiety and hunger signal to the subject is accomplished by direct conduction or transmission through unbroken skin at a zone adapted to communicate with a nerve, organ or muscle of the digestive system. Such zone will preferably approximate a position close to the nerve or nerve plexus onto which the signal is to be imposed.

In an alternate embodiment of the invention, the step of transmitting a gastrointestinal (i.e. satiety and hunger) signal and/or confounding gastrointestinal (i.e. satiety and hunger) signal and/or inverted satiety and hunger signal to the subject is accomplished by direct conduction via attachment of an electrode to the receiving nerve or nerve plexus. This requires a surgical intervention to physically attach the electrode to the selected target nerve.

In yet another embodiment of the invention, the step of transmitting a gastrointestinal signal and/or confounding gastrointestinal signal and/or inverted satiety and hunger signal to the subject is accomplished by transposing the waveform signal into a seismic form in a manner that allows the appropriate “nerve” to receive and obey the coded instructions of the seismic signal.

According to the invention, a single gastrointestinal signal or a plurality of gastrointestinal signals can be transmitted to the subject in conjunction with one another.

A single confounding gastrointestinal signal or a plurality of confounding gastrointestinal signals can be transmitted to the subject in conjunction with one another.

Similarly, a single inverted satiety or hunger signal or a plurality of inverted satiety or hunger signals can be transmitted to the subject in conjunction with one another.

A plurality of gastrointestinal (i.e. satiety and hunger) signals and confounding gastrointestinal signals and inverted satiety or hunger signals can also be generated and transmitted to the subject.

The system for treating eating disorders, in accordance with one embodiment of the invention, generally comprises (i) a processor adapted to generate at least one gastrointestinal signal that substantially corresponds to a neuro-electrical signal that is generated in the body and produces a gastrointestinal effect in the body and/or a confounding gastrointestinal signal and/or an inverted satiety or hunger signal, and (ii) a signal transmitter adapted to be in communication with the subject's body for transmitting gastrointestinal signals and inverted satiety and hunger signals and confounding gastrointestinal signals to the subject.

In another embodiment of the invention, the system for treating eating disorders comprises (i) at least a first food intake sensor adapted to monitor the food intake of a subject and provide at least a first food intake signal indicative of the food intake, (ii) a processor in communication with the food intake sensor adapted to receive the first food intake signal, the processor being further adapted to generate at least one gastrointestinal signal that substantially corresponds to a neuro-electrical signal that is generated in the body and produces a gastrointestinal effect in the body and/or a confounding gastrointestinal signal and/or an inverted satiety or hunger signal, and (iii) a signal transmitter adapted to be in communication with the subject's body for transmitting gastrointestinal signals and inverted satiety and hunger signals and confounding gastrointestinal signals to the subject.

### EXAMPLES

Methods of using the methods and systems of the invention will now be described in detail. The methods set forth herein are merely examples of envisioned uses of the methods and systems to control and/or limit food intake and thus should not be considered as limiting the scope of the invention.

### Example 1

A 45 year old female suffers from morbid obesity. She has been overweight since a first pregnancy, and her weight is now in excess of 200 percent of her ideal weight. She suffers from hypertension and sleep apnea, which her physician believes are directly related to her weight problem.

The patient consults with a physician and dietician to work out a diet and walking regimen for long-term weight loss. In coordination with this regimen, the patient has a neural stimulator implanted in her body, which embodies features of the invention. In this example, the stimulator is designed to generate and transmit confounding gastrointestinal signals and gastrointestinal signals that correspond to neuro-electrical signals that derive from the neck of the stomach, which elicit a feeling of fullness or satiety in the brain.

In this example, the patient monitors her weight weekly. It is expected that the patient will have periodic visits to her primary care physician for adjustment in the timing and duration of the neuro-electrical signals, and remain on the exercise and diet regimen during treatment.

### Example 2

A 50 year old sedentary, smoking male is diagnosed with chronic obstructive lung disease. His weight and limited lung function result in debilitating limitations on his mobility and lifestyle. His health status means that he is a very poor risk for invasive surgery, and previous attempts at weight loss have been ineffective.

The patient initially consults with a physician. The patient also receives extensive counseling and is advised to exercise as much as practical. As part of his treatment, the patient is prescribed a neural stimulator embodying features of the invention. The stimulator is installed in a minimally invasive procedure, and directly transmits generated inverted hunger signals and gastrointestinal satiety signals that produce a gastrointestinal effect in the patient's body, i.e. a feeling of fullness, to the vagus nerve with electrodes placed in the neck.

It is expected that the patient will have periodic visits to his primary care physician for adjustment in the timing and duration of the signals, and remain on the exercise and diet regimen during treatment.

As will be appreciated by one having ordinary skill in the art, the present invention provides numerous advantages. Among the advantages are the provision of a method and system for treating eating disorders having:

Enhanced effectiveness;

Reduced signal amplitude;

Reduced deleterious side effects;

More effective satiety effects; and

Less user discomfort.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for treating eating disorders, comprising the steps of:
generating at least one inverted hunger signal, said inverted hunger signal having at least a portion thereof that is substantially similar to a negative mirror image of at least a portion of at least one neuro-electrical signal that is generated in the body and induces a sensation of hunger in the body; and
transmitting said inverted hunger signal to said subject.

2. The method of claim 1, wherein said inverted hunger signal has a substantial portion that is substantially similar to a negative mirror image of a substantial portion of at least one neuro-electrical signal that is generated in the body and induces a sensation of hunger in the body.

3. The method of claim 1, wherein said inverted hunger signal is transmitted to said subject at predetermined time intervals.

4. The method of claim 1, wherein said inverted hunger signal is transmitted to said subject manually.

5. The method of claim 1, wherein said inverted hunger signal is transmitted to said subject at predetermined time intervals and manually.

6. A method for treating eating disorders, comprising the steps of:
generating at least one inverted hunger signal, said inverted hunger signal having at least a portion thereof that is substantially similar to a negative mirror image of at least a portion of at least one neuro-electrical signal that is generated in the body and induces a sensation of hunger in the body;
generating at least one satiety signal that substantially corresponds to at least one neuro-electrical signal that is generated in the body and induces a feeling of fullness in the body; and
transmitting said inverted hunger signal and said satiety signal to the subject.

7. The method of claim 6, wherein said inverted hunger signal is transmitted to said subject at first predetermined time intervals.

8. The method of claim 6, wherein said inverted hunger signal is transmitted to said subject manually.

9. The method of claim 6, wherein said satiety signal is transmitted to said subject at second predetermined time intervals.

10. The method of claim 6, wherein said satiety signal is transmitted to said subject manually.

11. The method of claim 6, wherein said inverted hunger signal and said satiety signal are transmitted to said subject substantially simultaneously at said first predetermined time intervals.

12. The method of claim 6, wherein said inverted hunger signal and said satiety signal are transmitted to said subject manually.

13. The method of claim 6, wherein said inverted hunger signal and said satiety signal are transmitted to said subject manually and at said first predetermined time intervals.

14. A method for treating eating disorders, comprising the steps of:
generating at least one inverted hunger signal, said inverted hunger signal having at least a portion thereof that is substantially similar to a negative mirror image of at least a portion of at least one neuro-electrical signal that is generated in the body and induces a sensation of hunger in the body;
generating at least one confounding gastrointestinal signal that is adapted to confound at least one neuro-electrical signal that is generated in the body and induces a sensation of hunger in the body;
generating at least one satiety signal that substantially corresponds to at least one neuro-electrical signal that is generated in the body and induces a feeling of fullness in the body, and
transmitting said inverted hunger signal and said confounding gastrointestinal signal and said satiety signal to said subject.

15. A method for treating eating disorders, comprising the steps of:
capturing at least one neuro-electrical signal that is generated in the body and induces a feeling of fullness in the body;
generating at least one satiety signal based on said neuro-electrical signal, said satiety signal substantially corresponding to said neuro-electrical signal;
generating at least one inverted hunger signal, said inverted hunger signal having at least a portion thereof that is substantially similar to a negative mirror image of at least a portion of at least one neuro-electrical signal that is generated in the body and induces a sensation of hunger in the body;
sensing food intake in a subject over at least a first period of time; and
transmitting said satiety signal and said inverted hunger signal to said subject.

16. The method of claim 15, wherein said inverted hunger signal is transmitted to said subject when said food intake of said subject exceeds a predetermined threshold level during said first period of time.

17. The method of claim 15, wherein said satiety signal is transmitted to said subject when said food intake of said subject exceeds a predetermined threshold level during said first period of time.

18. The method of claim 15, wherein said inverted hunger signal and said satiety signal are transmitted to said subject when said food intake of said subject exceeds a predetermined threshold level during said first period of time.

19. A method for treating eating disorders, comprising the steps of:
capturing at least one neuro-electrical signal that is generated in the body and induces a feeling of fullness in the body;
generating at least one satiety signal based on said neuro-electrical signal, said satiety signal substantially corresponding to said neuro-electrical signal;
generating at least one inverted hunger signal, said inverted hunger signal having at least a portion thereof that is substantially similar to a negative mirror image of at least a portion of at least one neuro-electrical signal that is generated in the body and induces a sensation of hunger in the body;
generating at least one confounding gastrointestinal signal that is adapted to confound at least one neuro-electrical signal that is generated in the body and induces a sensation of hunger in the body;
sensing food intake in a subject over at least a first period of time; and
transmitting said satiety signal and said inverted hunger signal and said confounding gastrointestinal signal to said subject.

20. The method of claim 19, wherein said inverted hunger signal is transmitted to said subject when said food intake of said subject exceeds a predetermined threshold level during said first period of time.

21. The method of claim 19, wherein said satiety signal is transmitted to said subject when said food intake of said subject exceeds a predetermined threshold level during said first period of time.

22. The method of claim 19, wherein said confounding gastrointestinal signal is transmitted to said subject when said food intake of said subject exceeds a predetermined threshold level during said first period of time.

23. A method for treating eating disorders, comprising the steps of:
capturing at least one neuro-electrical signal that is generated in the body and induces a feeling of fullness in the body;
generating at least one satiety signal based on said neuro-electrical signal, said satiety signal substantially corresponding to said neuro-electrical signal;
generating at least one confounding gastrointestinal signal that is adapted to confound at least one neuro-electrical signal that is generated in the body and induces a sensation of hunger in the body;
sensing food intake in a subject over at least a first period of time; and
transmitting said satiety signal and said confounding gastrointestinal signal to said subject.

24. A method for treating eating disorders, comprising the steps of:
generating at least one confounding gastrointestinal signal that is adapted to confound at least one neuro-electrical signal that is generated in the body and induces a sensation of hunger in the body;
generating at least one satiety signal that substantially corresponds to at least one neuro-electrical signal that is generated in the body and induces a feeling of fullness in the body;
transmitting said confounding gastrointestinal signal and said satiety signal to said subject.

25. A method for treating eating disorders, comprising the steps of:
generating at least one inverted satiety signal, said inverted satiety signal having at least a portion thereof that is substantially similar to a negative mirror image of at least a portion of at least one neuro-electrical signal that is generated in the body and induces a feeling of fullness in the body; and
transmitting said inverted satiety signal to said subject.

26. The method of claim 25, wherein said inverted satiety signal has a substantial portion that is substantially similar to a negative mirror image of a substantial portion of at least one neuro-electrical signal that is generated in the body and induces a feeling of fullness in the body.

27. The method of claim 25, wherein said inverted satiety signal is transmitted to said subject at predetermined time intervals.

28. The method of claim 25, wherein said inverted satiety signal is transmitted to said subject manually.

29. The method of claim 25, wherein said inverted satiety signal is transmitted to said subject at predetermined time intervals and manually.

30. A method for treating eating disorders, comprising the steps of:
generating at least one inverted satiety signal, said inverted satiety signal having at least a portion thereof that is substantially similar to a negative mirror image of at least a portion of at least one neuro-electrical signal that is generated in the body and induces a feeling of fullness in the body;
generating at least one hunger signal that substantially corresponds to at least one neuro-electrical signal that is generated in the body and induces a sensation of hunger in the body;
transmitting said inverted satiety signal and said hunger signal to said subject.

31. The method of claim 30, wherein said inverted satiety signal is transmitted to said subject at first predetermined time intervals.

32. The method of claim 30, wherein said inverted satiety signal is transmitted to said subject manually.

33. The method of claim 30, wherein said hunger signal is transmitted to said subject at second predetermined time intervals.

34. The method of claim 30, wherein said hunger signal is transmitted to said subject manually.

35. The method of claim 30, wherein said inverted satiety signal and said hunger signal are transmitted to said subject substantially simultaneously at said first predetermined time intervals.

36. The method of claim 30, wherein said inverted satiety signal and said hunger signal are transmitted to said subject manually.

37. The method of claim 30, wherein said inverted satiety signal and said hunger signal are transmitted to said subject manually and at said first predetermined time intervals.

38. A method for treating eating disorders, comprising the steps of:
generating at least one inverted satiety signal, said inverted satiety signal having at least a portion thereof that is substantially similar to a negative mirror image of at least a portion of at least one neuro-electrical signal that is generated in the body and induces a feeling of fullness in the body;
generating at least one confounding gastrointestinal signal that is adapted to confound at least one neuro-electrical signal that is generated in the body and induces a feeling of fullness in the body;
generating at least one hunger signal that substantially corresponds to at least one neuro-electrical signal that is generated in the body and induces a sensation of hunger in the body; and
transmitting said inverted satiety signal and said confounding gastrointestinal signal and said hunger signal to said subject.

39. A method for treating eating disorders, comprising the steps of:
capturing at least one neuro-electrical signal that is generated in the body and induces a sensation of hunger in the body;
generating at least one hunger signal based on said neuro-electrical signal, said hunger signal substantially corresponding to the neuro-electrical signal;
generating at least one inverted satiety signal, said inverted satiety signal having at least a portion thereof that is substantially similar to a negative mirror image of at least a portion thereof that is substantially similar to a negative mirror image of a portion of at least one neuro-electrical signal that is generated in the body and induces a feeling of fullness in the body;
sensing food intake in a subject over at least a first period of time; and
transmitting said hunger signal and said inverted satiety signal to said subject.

40. The method of claim 39, wherein said inverted satiety signal is transmitted to said subject when said food intake of said subject falls below a predetermined threshold level during said first period of time.

41. The method of claim 39, wherein said hunger signal is transmitted to said subject when said food intake of said subject falls below a predetermined threshold level during said first period of time.

42. The method of claim 39, wherein said inverted satiety signal and said hunger signal are transmitted to said subject when said food intake of said subject falls below a predetermined threshold level during said first period of time.

43. A method for treating eating disorders, comprising the steps of:
capturing at least one neuro-electrical signal that is generated in the body and induces a sensation of hunger in the body;
generating at least one hunger signal based on said neuro-electrical signal, said hunger signal substantially corresponding to said neuro-electrical signal;
generating at least one inverted satiety signal, said inverted satiety signal having at least a portion thereof that is substantially similar to a negative mirror image of at least a portion of at least one neuro-electrical signal that is generated in the body and induces a feeling of fullness in the body;
generating at least one confounding gastrointestinal signal that is adapted to confound at least one neuro-electrical signal that is generated in the body and induces a feeling of fullness in the body;
sensing food intake in a subject over at least first period of time; and
transmitting said hunger signal and said inverted satiety signal and said confounding gastrointestinal signal to said subject.

44. The method of claim 43, wherein said inverted satiety signal is transmitted to said subject when said food intake of said subject falls below a predetermined threshold level during said first period of time.

45. The method of claim 43, wherein said hunger signal is transmitted to said subject when said food intake of said subject falls below a predetermined threshold level during said first period of time.

46. The method of claim 43, wherein said confounding gastrointestinal signal is transmitted to said subject when said food intake of said subject falls below a predetermined threshold level during said first period of time.

47. A method for treating eating disorders, comprising the steps of:
capturing at least one neuro-electrical signal that is generated in the body and induces a sensation of hunger in the body;
generating at least one hunger signal based on said neuro-electrical signal, said hunger signal substantially corresponding to said neuro-electrical signal;
generating at least one confounding gastrointestinal signal that is adapted to confound at least one neuro-electrical signal that is generated in the body and induces a feeling of fullness in the body;
sensing food intake in a subject over at least a first period of time; and
transmitting said hunger signal and said confounding gastrointestinal signal to said subject.

48. A method for treating eating disorders, comprising the steps of:
generating at least one confounding gastrointestinal signal that is adapted to confound neuro-electrical signals that are generated in the body and induce a feeling of fullness;
generating at least one hunger signal that substantially corresponds to at least one neuro-electrical signal that is generated in a body and induces a sensation of hunger in the body; and
transmitting said confounding gastrointestinal signal and said hunger signal to said subject.
